(19) United States
(12) Patent Application Publication
(10) Pub. No.: US 2004/0116887 A1
(43) Pub. Date: Jun. 17, 2004

(54) Disposable undergarment with a gathered crotch member and method for the manufacture thereof

(76) Inventors: Russell E. Thorson, Appleton, WI (US); Paul T. Van Gompel, Hortonville, WI (US); Jacqueline A. Gross, Neenah, WI (US)

Correspondence Address: BRINKS HOFER GILSON & LIONE, P.O. BOX 10395, CHICAGO, IL, 60610, US

(21) Appl. No.: 10/318,329

(22) Filed: Dec. 12, 2002

(51) Int. Cl. A61F 13/15; A61F 13/20

(52) U.S. Cl. 604/385.22

(57) ABSTRACT

A method of manufacturing a disposable undergarment includes elongating a body panel material in a first direction to an elongated condition, attaching a crotch member to the body panel material while the body panel material is in the elongated condition, and retracting the body panel material in the first direction from the elongated condition to a relaxed condition. A disposable undergarment, and a method for using the garment, are also provided.

### BACKGROUND

The present invention relates generally to disposable undergarments, and in particular, to an undergarment having a gathered crotch member, and to the methods for the manufacture and use thereof.

Disposable undergarments can be configured in many different forms. For example, disposable absorbent garments can be configured as a pant-type, pull-on garment, or as a diaper-type product that is drawn up between the legs and fastened about the waist with various fastening systems. In some configurations, the garment is formed from a crotch member attached to a body panel, which may be elongateable. Typically, the crotch member is secured to the body panel when the body panel is in a relaxed condition. In addition, the crotch member is often secured across the entire width of the portion of the crotch member that overlaps the body panel. As such, the crotch member, which is often non-elastic, can reduce or otherwise impede the elongation of the body panel, thereby reducing its ability to conform to the body of the user. In addition, the crotch member typically is not able to conform to the body of the user independently of the body panel, and can therefore distort the panels when fitted to a user.

Therefore the need remains for an improved undergarment that conforms to the body of the user during use without interference from a crotch portion thereof, and for improved methods and assemblies for manufacturing such undergarments.

### SUMMARY

Briefly stated, in one embodiment described below, a method of manufacturing an undergarment includes elongating a body panel material in a first direction to an elongated condition, attaching a crotch member to the body panel material while the body panel material is in the elongated condition, and retracting the body panel material in the first direction from the elongated condition to a relaxed condition. In one embodiment, the crotch member is gathered as the body panel material is retracted from the elongated condition to the relaxed condition. Also in one exemplary embodiment, the crotch member includes an absorbent component.

In one embodiment, the method includes elongating a first body panel material in a first direction to a first elongated condition, elongating a second body panel material in the first direction to a second elongated condition, and attaching an absorbent insert to the first and second panel materials at a plurality of attachment locations while the first and second body panel materials are in the first and second elongated conditions respectively. The attachment locations are spaced apart in the first direction. The method further includes retracting the first and second body panel materials in the first direction from the first and second elongated conditions to first and second relaxed conditions respectively.

In another aspect, a disposable undergarment includes a body panel and a crotch member attached to the body panel. The body panel is elongateable in a first direction between at least a relaxed condition and an elongated condition. The crotch member is elongateable in the first direction between at least a gathered condition and an ungathered condition. The crotch member is in the gathered condition when the body panel is in the relaxed condition, and the crotch member is in the ungathered condition when the body panel is in the elongated condition.

In yet another aspect, a method of using a disposable undergarment includes providing an enlongateable body panel in a relaxed condition and a crotch member attached to the body panel. The crotch member is in a gathered condition when the body panel is in the relaxed condition. The method further includes elongating the body panel in a first direction from the relaxed condition to an elongated condition and thereby elongating the gathered crotch member in the first direction.

The various presently preferred embodiments provide significant advantages over other disposable undergarments, and methods for the manufacture and use thereof. For example, the gathered crotch member does not restrict the elongation of the body panel, but rather can be elongated therewith. Accordingly, as the body panel is applied to the user, the body panel material can elongate and conform to the body of the user, with the crotch member elongating with the body panel. This configuration provides for a body conforming fit of the body panel and also a snug fit of the crotch member, for example an absorbent insert, to the body of the user.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

Many of the features and dimensions portrayed in the drawings, and in particular the presentation of layer thicknesses and the like, have been somewhat exaggerated for the sake of illustration and clarity.

FIG. 1 is a plan view of a first embodiment of an absorbent garment taken from the bodyside thereof.

FIG. 2 is a schematic illustration of a cross-sectional view of the absorbent garment taken along line 2-2 in FIG. 1.

FIG. 3 is a schematic illustration of a cross-sectional view of the absorbent garment taken along line 3-3 in FIG. 1.

FIG. 4 is a schematic illustration of a cross-sectional view of the absorbent garment taken along line 4-4 in FIG. 1.

FIG. 4A is a schematic illustration of a cross-sectional view of an alternative embodiment of the absorbent garment taken along line 4-4 in FIG. 1.

FIG. 4B is a schematic illustration of a cross-sectional view of an alternative embodiment of the absorbent garment taken along line 4-4 in FIG. 1.

FIG. 4C is a schematic illustration of a cross-sectional view of an alternative embodiment of the absorbent garment taken along line 4-4 in FIG. 1.

FIG. 5 is a schematic illustration of one embodiment of manufacturing assembly used to assemble an undergarment.

### DETAILED DESCRIPTION OF THE PRESENTLY PREFERRED EMBODIMENTS

It should be understood that the term “longitudinal,” as used herein, means of or relating to length or the lengthwise direction, for example the lengthwise direction 500 of the undergarment shown in FIG. 1. The term “laterally,” as used herein means situated on, directed toward or running from side to side, for example the side to side direction 502 of the undergarment shown in FIG. 1. The term “first direction” generally refers to a path, line or course rather than a vector, and applies equally to opposite orientation along the path, line or course, including for example and without limitation movement along a path, line or course in both directions. Likewise, the term “second direction” generally refers to a path, line or course rather than a vector (not orientation dependent), and includes for example and without limitation movement along a path, line or course in both directions. In one example, the first direction is defined by and refers to one of the longitudinal and lateral directions, while the second direction refers to the other of the longitudinal and lateral directions.

The term “bodyside” should not be interpreted to mean in contact with the body of the user, but rather simply means the side that would face toward the body of the user, regardless of whether an undergarment is actually being worn by the user and regardless of whether there are or may be intervening layers between the component and the body of the user. Likewise, the term “garment side” should not be interpreted to mean in contact with the garments of the user, but rather simply means the side that faces away from the body of the user, and therefore toward any outer garments that may be worn by the user, regardless of whether the undergarment is actually being worn by a user, regardless of whether any such outer garments are actually worn and regardless of whether there may be intervening layers between the component and any outer garment.

The term “machine direction” means the direction of flow as the various members and webs progress along the fabrication line and process. It should be understood that various separate members or webs can each be traveling in a machine direction, but with the various machine directions not necessarily being parallel or oriented in the same direction. For example, a first component such as a web may be traveling a first machine direction, which is substantially perpendicular to the travel of another component, such as an absorbent insert, in a second machine direction.

The term “cross direction” means the direction substantially perpendicular to the machine direction.

The term “downstream” means that one item is positioned more closely to the output or finished product end of the machine and/or process relative to another item. Conversely, the term “upstream” means that an item is positioned more closely to the input end of the machine or process relative to another item. For example, the output end is downstream of the input end, and vice versa, the input end is upstream of the output end.

The phrases “removeably attached,” “removeably attaching,” “removeably connected,” “removeably engaged,” “releasably attached,” “releasably connected,” or “releasably engaged,” and variations thereof, refers to two or more elements being connected or connectable such that the elements tend to remain connected absent a separation force applied to one, both or all of the elements, and where the elements are capable of being separated upon the application of a separation force. The required separation force is typically beyond that encountered while wearing the absorbent garment.

The phrases “fixedly secured,” “fixedly engaged,” “fixedly attached,” “fixedly connected,” and variations thereof, refers to two or more elements being connected or connectable such that they are not disconnected or otherwise separated, and are not intended to be separated or disconnected, during the normal operation and use of the absorbent garment.

The term “web” refers to a continuous stream of material, whether made from one or more layers or substrates, and regardless of whether it may have non-continuous, discrete items disposed thereon.

The terms “connecting,” “coupled,” “attached,” and “secured,” and variations thereof, broadly covers two or more items being directly connected one to the other, or by way of one or more intervening members or components.

Referring to FIGS. 1-4, an undergarment 2 includes a first, front body panel 4 and a second, rear body panel 6. The term “body panel” refers to the portion(s) of the undergarment, whether made of one or more layers or substrates or of one or more pieces or components, that is/are fitted circumferentially around at least the waist region of the user, including for example the user's lower back, buttocks, hips and abdomen. The first and second body panels each have an inner, bodyside surface 10 and an outer, garment side surface 12. The first, front body panel 4 has a first edge 14 having a crotch portion 16 and a leg opening portion 18 and a second terminal edge 20 that is linear but can assume other shapes. Likewise, the second, rear body panel 6 has a first edge 22 having a crotch portion 24 and a leg opening portion 26 and a second terminal edge 28, which is linear but can assume other shapes. Each of the first and second body panels have an outboard side edge 30, 32 formed along the outer periphery of the opposite side portions of the first and second body panel. It should be understood that the outboard side edges of the front and rear body panels could have different lengths relative to each other.

In one embodiment, one or more, and preferably a plurality, meaning two or more, elastic elements are secured to each of the first and second body panels. In one embodiment, a plurality of elastic elements are spaced across substantially the entire waist portion of the front and rear body panel, although they may be spaced across a lesser length. For example, elastic elements can extend along the upper waist portion and along the lower terminal edge defining in part a leg opening.

In one embodiment, the front body panel has a “non-elasticized” area wherein there are no elastic elements, or other elastic or elastomeric backing members, incorporated therein or making up any portion of the thickness or cross-section of the body panel at that area. It should be understood, that in various embodiments, one or more separate waist bands, with or without elastic elements, can be secured to one or both of the rear and front body panels, for example along the upper terminal edges 20, 28 thereof. Likewise, one or more separate leg bands can be secured to one or both of the rear and front body panels along the leg open portions 18, 26 adjacent the leg openings 34. Alternatively, one or both of the body panels can be formed without any elastic elements.

The various waist and leg elastic elements can be formed from rubber or other elastomeric materials. One suitable material is a LYCRA® elastic material. For example, the various elastic elements can be formed of LYCRA® XA Spandex 540, 740 or 940 decitex T-127 or T-128 elastics available from E.I. duPont De Nemours and Company, having an office in Wilmington, Del.

Referring to FIGS. 1, 3 and 4-4C, each body panel 4, 6 is formed as a composite, or laminate material, otherwise referred to as substrates or laminates, with one or more elastic elements 36 sandwiched therebetween. The elastic element can be formed from a film, which can be apertured, ribbons, strands or other types of elastic elements. In one embodiment, two or more layers 40 are bonded to the elastic element 36, and/or each other, with various adhesives, such as hot melt, or by other techniques, including for example and without limitation ultrasonic bonding and heat pressure sealing. In one embodiment, the two layers are made of a non-woven material such as a spunbond material, a bonded carded material or other known materials. It should be understood that the body panels can be made of a single layer or substrate of non-woven material, or can be comprised of more than two layers or substrates. Of course, it should be understood that other knitted or woven fabrics, non-woven fabrics, elastomeric materials, polymer films, laminates and the like can be used to form one or more of the body panel layers. The term “non-woven” web or material, as used herein, means a web having a structure of individual fibers or filaments that are interlaid, but not in an identifiable manner and without the aid of textile weaving or knitting, as in a knitted or woven fabric.

In one embodiment, the body panel material can be secured to the elastic element(s), such as an elastomeric layer or elastic strands or ribbons, which have been elongated and retracted, such that the material is gathered when the elastic element(s) are relaxed. Alternatively, the material can be gathered and laminated to non-elongated elastic elements. In one embodiment, the body panel includes a gathered elastic laminate made from nonwoven base sheets bonded with elongated elastic elements sandwiched therebetween.

In various embodiments, the body panel material may be substantially permeable to air or substantially impermeable to air. The body panel material also may be substantially liquid-permeable or substantially liquid-impermeable. In particular arrangements, the body panel material may be substantially nonelastomeric. In other aspects, the body panels can include an elastomeric material that is elastomerically stretchable at least along one or both of the lateral article width and the longitudinal article length. Examples of such elastomeric composite materials can include a vertical filament laminate (VFL), neck-bonded-laminate (NBL), a stretch-bonded-laminate (SBL), a necked-stretch bonded laminate (NSBL) or a necked-thermal laminate, or the like, as well as combinations thereof. Exemplary NBL, SBL, and NSBL materials are described in U.S. Pat. Nos. 5,226,992, 4,981,747, 4,965,122, 5,336,545, 5,385,775, 5,414,470, 4,720,415, 4,789,699, 4,781,966, 4,657,802, 4,652,487, 4,655,760, 5,116,662 and 5,114,781, all of which are hereby incorporated herein by reference. Exemplary VFL materials are described in U.S. Provisional Patent Application Serial No. 60/204,307, filed May 15, 2000 and entitled “Method and Apparatus for Producing Laminated Articles,” and PCT application WO 01/88245 A2, both assigned to Kimberly-Clark Worldwide, Inc., the Assignee of the present application, with the entire disclosures of both being hereby incorporated herein by reference. Such laminates can provide an improved combination of cloth-like feel and elastomeric stretchability. The body panels can be composed of materials that are elastic or elastomeric and exhibit biaxial stretch characteristics or MD/CD stretch characteristics, or that are extensible composites. Additional waist and leg elastic elements can be added to, but are not necessarily required by, the body panels.

As shown in FIG. 1, in one embodiment, the entirety of the body panels 4, 6 are elasticized, such that the entire body panel conforms to the body of the user without any spacing between the body panel and the user's body, and without the attendant bulkiness of a non-elasticized material.

In one embodiment, the body panels are breathable, cloth-like, multi-directional nonwoven laminates with stretch and/or extensible properties. In one embodiment, the non-woven layers are pre-necked, for example between about 10% and about 80%, in the longitudinal direction, which provides extensibility in the longitudinal direction with minimum force.

The terms “extensible,” “extensibility,” and variations thereof as used herein means capable of being extended, and providing a selected elongation, for example between about 5% and about 70%, when subjected to an applied tensile force. The body panel also is capable of providing a selected, sustained deformation when subjected to an applied tensile force and then allowed to relax for a selected time period beginning immediately after removal of the tensile force. The sustained deformation is a substantially permanent deformation. The selected elongation and sustained deformation occur at least along the longitudinal direction of the garment, although it should be understood that it also could occur along the lateral direction, or both. Various extensible materials, and other acceptable materials that can be used for the body panels are described for example in U.S. Pat. No. 6,217,563, issued Apr. 17, 2001 to Kimberly-Clark Worldwide, Inc., the same Assignee as the present application, the entire disclosure of which is hereby incorporated herein by reference.

The extensibility of the exemplary non-woven material provides an increase in surface area without the retractive force of elastomeric materials. In one embodiment, body panel is extensible in at least the cross-direction, or longitudinal direction, with the material providing an elongation-of at least about 1 cm when subjected to a tensile force of 11.8 grams per cm. In addition, the body panel provides a substantially permanent deformation of at least about 20% when it is subjected to a tensile force of 19.70 grams per cm and is then allowed to relax under a zero applied force for a period of 1 minute. Of course, it should be understood that the body panel can also be made extensible in the lateral direction.

In one embodiment, the front and rear body panels 4, 6 are made of non-woven laminates of two layers of longitudinally extensible 0.60 osy polypropylene spunbond material with elongated strands of Lycra® elastic sandwiched between the spunbond layers and thereafter adhesively bonded. In particular, the body panel material is necked in the cross direction. As used herein, the term “necked,” and variations thereof, refers to any material that has been constricted in at least one dimension by applying a tensioning force in a direction that is perpendicular to the desired direction of neck-down. Processes that may be used to constrict a material in such a manner include, for example and without limitation, drawing processes. The elastics are then elongated in the machine direction and secured to the body panel material. The elastics are then allowed to retract so as to gather the necked spunbond material in the machine direction (lateral body panel direction) thereby creating an elastically gathered non-woven body panel with longitudinal extensibility. The term “gather,” and variations thereof, as used herein means puckered, or contracted into folds or wrinkles, which should be understood as including micro-pleats. In this way, the body panel can be elongated in both the longitudinal and lateral direction to conform to the body of the user when the garment is applied thereto. In particular, as the user pulls the garment up over their hips, the non-woven laminate body panels stretch in the lateral direction while the leg regions of the front and rear body panels conform to the crotch and body lines of the user. At the same time, the body panel material extends in the longitudinal direction to conform to the buttocks and stomach of the user. The extensibility of the body panels follows the natural curvature of user's body to provide conformance thereto. As the body panel extends in the longitudinal direction, the spacing between the laterally extending elastic elements, incorporated in one embodiment, will increase.

The body panel 4, 6 non-woven material is substantially hydrophobic, which may optionally be treated with a surfactant or otherwise processed to impart a desired level of wettability and hydrophilicity. In one particular embodiment of the invention, the body panel is a nonwoven, wire-weave spunbond polypropylene fabric composed of about 1.6 denier fibers formed into a web having a basis weight of about 0.6 osy. One suitable non-woven material is the Corinth 0.60 osy, 1.6 dpf wireweave, nonwettable Metallocene (EXXON ACHIEVE 2854 PP) spunbond material manufactured by Kimberly-Clark Corporation, the assignee of the present application.

Referring to FIG. 1, a crotch member 50 connecting the front and rear body panels 4, 6 can be folded such that the side edges 30, 32 of the front and rear body panels 4, 6 are aligned wherein they can be fixedly secured at a seam. The seam can be formed by bonding, sewing or otherwise attaching the side edges. Alternatively, the product can remain “open,” wherein the body panels are releasably secured with one or more fastening members as explained below.

In one embodiment, the garment includes a combination of side edges that are secured to form a seam and fastening members that allow the fit of the undergarment to be adjusted. For example, in one embodiment, fastening members are attached to the front body panel and extend inboard relative to the outboard side edge 30 of the front body panel 4 from an attachment location, which is spaced inboard from the side edge. A landing member can be formed on or secured to the body panel to receive a refastenable portion of the fastening member. One or more lines of weakness can be provided along the front or rear body panel such that one or both of the body panels are breakable. The lines of weakness can comprise a perforation or other series of cuts, a thinning, breakage or separation of material, or a strip of a different kind of material bridging portions of the body panel that is more easily torn or broken than the other material thereof, which allow a user or the manufacturer to separate portions of the body panel. For example, the undergarment can be broken along the lines of weakness after the garment is applied to a user, or beforehand. The fastening members are secured to the garment-side surface of the body panel.

It should be understood that, in other embodiments, the fastening members can be secured to the rear body panel and engage the front body panel or, conversely, can be secured to the front body panel and engage the rear body panel, for example along at least a portion that is not elasticized. The fastening members are fixedly secured to the outer, garment-side surface of the front and/or rear body panels, and releasably engage the outer, garment-side surface of the front and/or rear body panels, although it should be understood that the fastening members could be fixedly secured to an inner body-side surface of front and/or rear body panels and releasably engage an inner, body-side surface of the front and/or rear body panels.

When incorporated into a disposable absorbent undergarment, the fastening members include a refastenable portion, such as an array of hook members, adhesives, such as pressure sensitive adhesives, buttons, zippers, snaps and other releasable and reattachable fastening devices. In various embodiments, the fastening member includes one, two or more than two tab members. In one embodiment, the fastening members comprise a carrier member, which is fixedly secured to the side portions of the front body panel with adhesive bonds, sonic bonds, thermal bonds, pinning, stitching or other known types of attachment. In alternative embodiments, the fastening members can be fixedly secured to the rear body panel or to one or both of the front and rear body panels, for example, at the seam, as explained above.

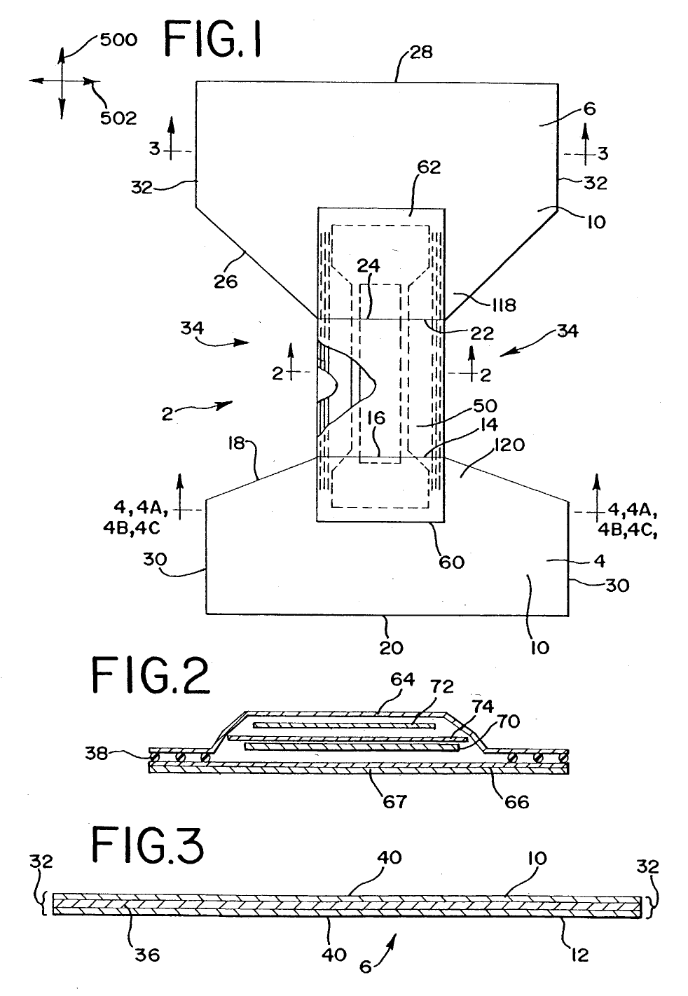

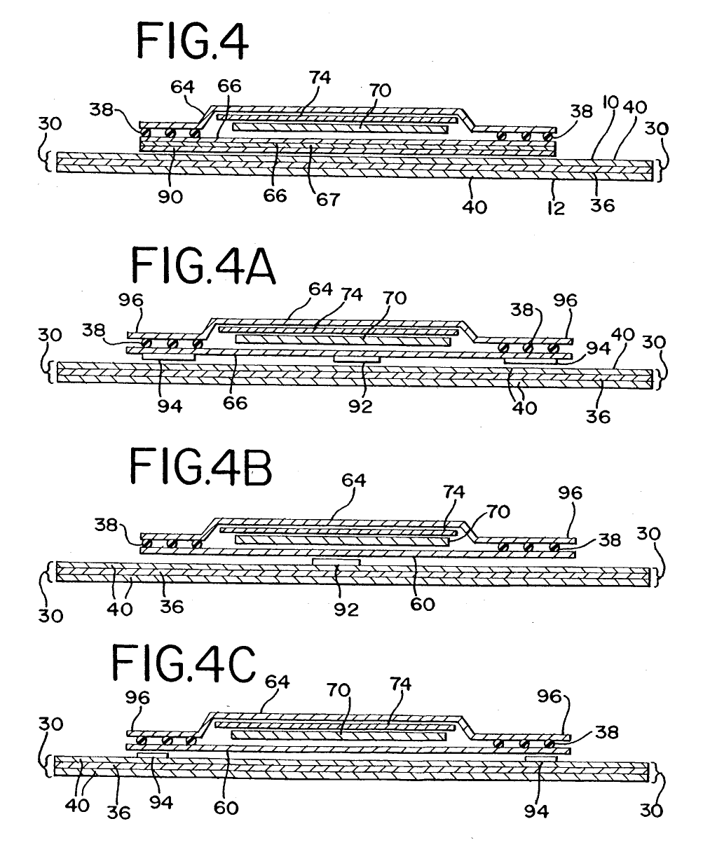

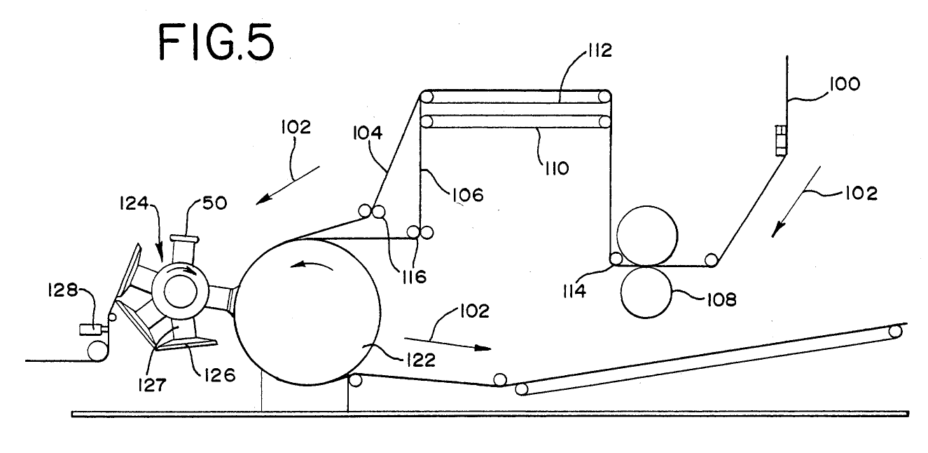

Referring to FIGS. 1-4C, in one embodiment, the undergarment is disposable and is also configured as an absorbent undergarment. In the absorbent garment, the crotch member is configured as an absorbent insert 50 having first and second opposed terminal end edges 60, 62. In one embodiment, the absorbent insert 50 includes a substantially liquid permeable topsheet 64, or liner, and a substantially liquid impermeable backsheet 66. A retention portion 70 is disposed or sandwiched between the topsheet and the backsheet, which are connected. In one embodiment, shown in FIGS. 2 and 4, an outer cover 67, such as a non-woven material, is secured to the backsheet 66. In other embodiments, shown for example in FIGS. 4A-4C, the outer cover is omitted. In yet another embodiment, the outer cover extends the length of the garment and is disposed over the body panels and crotch member. In yet another embodiment, the outer cover extends the length of and is secured only to the portion of the crotch member that is exposed between and bridges the front and rear body panels. In yet another embodiment, the outer cover, which extends the length of the garment, makes up one of the layers of the front and rear body panels. In various embodiments, the outer cover is made of the same material as the body panels, including for example a non-woven material.

The topsheet 64, backsheet 66 and other components of the absorbent insert 50 can be joined for example with adhesive bonds, sonic bonds, thermal bonds, pinning, stitching or any other attachment techniques known in the art, as well as combinations thereof. For example, a uniform continuous layer of adhesive, a patterned layer of adhesive, a sprayed pattern of adhesive or any array of lines, swirls or spots of construction bonds may be used to join the topsheet and backsheet, or any of the other components described herein. One or more crotch elastic members 38 are sandwiched between the top sheet 64 and backsheet 66. It should be understood that the term “absorbent insert” refers to any material or assembly capable of absorbing liquids or bodily exudates, and may be comprised of a single material or component, for example a retention portion, or can be formed as a composite of several components. It should also be understood that the term “crotch member” refers to any member made of any material, including for example and without limitation those described herein with respect to the body panels and absorbent inserts, and is not limited to absorbent inserts and/or materials. For example, the crotch member may be made of one or more layers of a non-woven material.

In one embodiment, additional layers, including for example, a intake/distribution layer 72, otherwise referred to as a surge layer or transfer layer, are also incorporated into the absorbent insert. The surge layer does not run the entire length of the absorbent insert and is shorter than the retention portion. The topsheet can be indirectly joined to the backsheet by affixing the topsheet to intermediate layers, such as the surge layer or retention portion, which in turn is affixed to the backsheet. The absorbent insert also may include barrier cuffs, or leakage control shields, formed along the opposite longitudinally extending edges of the absorbent composite.

In one embodiment, the backsheet is liquid impermeable, but may be liquid permeable, e.g., when an additional barrier layer is used with the retention portion. For example, in one embodiment, the backsheet can be made from a thin plastic film, or other flexible, substantially liquid-impermeable material. As used herein, the term “flexible” means a material that is compliant and which will readily conform to the general shape and contour of the body of the user. The backsheet prevents various bodily fluids and exudates from wetting or otherwise contaminating various bedding or outer garments worn by the user over the absorbent garment. In particular, the backsheet can include a film, such as a polyethylene film, having a thickness of from about 0.012 mm to about 0.051 mm.

In various constructions, the topsheet can comprise various woven or nonwoven materials. For example, the topsheet can be composed of a meltblown or spunbonded web of desired fibers, and may also be a bonded-carded web. For example, the topsheet can be made of a substantially hydrophobic material, and the hydrophobic material may optionally be treated with a surfactant or otherwise processed to import a desired level of wettability and hydrophilicity. In one particular embodiment of the invention, the topsheet is a nonwoven, spunbond polypropylene fabric composed of about 2.8-3.2 denier fibers formed into a web having a basis weight of about 22 gsm and density of about 0.06 gm/cc. The fabric can be surface treated with an operative amount of surfactant, such as about 0.28% Triton X-102 surfactant. The surfactant can be applied by any conventional means, such as spraying, printing, brush coating or the like.

In various constructions, the backsheet can comprise a woven or nonwoven fibrous web layer, which is treated or constructed, partially or wholly, to impart the desired levels of liquid impermeability to selected regions that are adjacent to or proximate the absorbent retention portion. For example, the backsheet may include a gas-permeable, nonwoven fabric layer laminated to a polymer film layer which may or may not be gas-permeable. Other examples of fibrous, cloth-like backsheet materials can comprise a stretch thinned or stretch thermal laminate material composed of a 0.6 mil (0.015 mm) thick polypropylene cast film and a 0.7 ounce per square yard (23.8 gsm) polypropylene spunbond material (2 denier fibers). A material of this type has been employed to form the outercover of a HUGGIES® Ultratrim Disposable Diaper, which has been commercially available from Kimberly-Clark Corporation. The backsheet can provide the outercover of the article, particularly in the crotch region. Optionally, however, the article may include a separate outercover component member, as disclosed herein, which is additional to the backsheet. The outercover can be joined, for example, to one or more of the absorbent composite and/or body panels as explained above.

The backsheet may include a micro-porous, “breathable” material which permits gases, such as water vapor, to escape from the absorbent garment while substantially preventing liquid exudates from passing through the backsheet. For example, the breathable backsheet may be composed of a microporous polymer film or a nonwoven fabric which has been coated or otherwise modified to impart a desired level of liquid impermeability. For example, a suitable microporous film can be a PMP-1 material, which is available from Mitsui Toatsu Chemicals, Inc., a company having offices in Tokyo, Japan; or an XKO-8044 polyolefin film available from 3M Company of Minneapolis, Minn. The backsheet may also be embossed or otherwise provided with a pattern or matte finish to exhibit a more aesthetically pleasing appearance.

In various configurations of the invention, where a component, such as the backsheet is configured to be permeable to gas while having a resistance and limited permeability to aqueous liquid, the liquid resistant component can have a construction which is capable of supporting a selected hydrohead of water substantially without leakage therethrough. A suitable technique for determining the resistance of a material to liquid penetration is Federal Test Method Standard FTMS 191 Method 5514, 1978, or an equivalent thereof.

In one embodiment, the backsheet is sufficiently impermeable to liquid and semi-liquid materials to substantially prevent the undesired leakage of waste materials, defined as exudates, including for example urine and feces. For example, the backsheet member can desirably support a hydrohead of at least about 45 centimeters (cm) substantially without leakage. The backsheet member can alternatively support a hydrohead of at least about 55 cm, and optionally, can support a hydrohead of at least about 60 cm, or more, to provide improved benefits.

The backsheet and/or outercover also can be extensible. In one embodiment, the backsheet and/or outercover is capable of providing an elongation of at least about 1 cm when subjected to a tensile force of 11.8 g/cm, and further provides a substantially permanent deformation of at least about 20% when subjected to a tensile force of 19.70 g/cm and is then allowed to relax under a zero applied force for a period of 1 minute.

For example, the extensible member can be composed of a necked fiber, a creped fiber, a micro-pleated fiber, polymer films or the like, as well as combinations thereof. The fabrics may be woven or nonwoven materials, such as spunbond fabrics. One example of a suitable extensible material is a 60% necked, polypropylene spunbond having a basis weight of about 1.2 osy.

The backsheet and/or outercover also can be expandable, for example when it has one or more folds, e.g., one or more z-folds (not shown), or can be both extensible and expandable. The term expandable as used herein means to enlarge or to increase the extent or area, lateral and/or longitudinal, thereof, e.g., by unfolding one or more folds.

The retention portion 70 is made of an absorbent material, which can be any material that tends to swell or expand as it absorbs exudates, including various liquids and/or fluids excreted or exuded by the user. For example, the absorbent material can be made of airformed, airlaid and/or wetlaid composites of fibers and high absorbency materials, referred to as superabsorbents. Superabsorbents typically are made of polyacrylic acids, such as FAVOR 880 available from Stockhausen, Inc. of Greensboro, N.C. The fibers can be fluff pulp materials, such as Alliance CR-1654, or any combination of crosslinked pulps, hardwood, softwood, and synthetic fibers. Airlaid and wetlaid structures typically include binding agents, which are used to stabilize the structure. In addition, various foams, absorbent films, and superabsorbent fabrics can be used as an absorbent material. Various acceptable absorbent materials are disclosed in U.S. Pat. No. 5,147,343 for Absorbent Products Containing Hydrogels With Ability To Swell Against Pressure, U.S. Pat. No. 5,601,542 for Absorbent Composite, and U.S. Pat. No. 5,651,862 for Wet Formed Absorbent Composite, all of which are hereby incorporated herein by reference. Furthermore, the proportion of high-absorbency particles can range from about 0 to about 100%, and the proportion of fibrous material from about 0 to about 100%. Additionally, high absorbency fibers can be used such as Oasis type 121 and type 122 superabsorbent fibers available from Technical Absorbent Ltd., Grimsby, Lincolnshire, United Kingdom.

The retention portion can be made of a single or dual layer of absorbent material. The retention portion has an hour-glass shape with enlarged end regions. Alternatively, the retention portion can include a folded or multi-layered configuration. The retention portion has a length substantially equal to, or slightly shorter than, the length of the absorbent insert. The retention portion can include one or more barrier layers attached to the absorbent material. In one embodiment, an upper tissue substrate is disposed adjacent the retention portion. Alternatively, a lower tissue substrate 74 can be disposed adjacent an opposite side of the retention portion, or the tissue can completely envelope the retention position.

Referring to FIGS. 1 and 4-4C, the opposite garment side of the end regions of the crotch member 50, and in particular, the outer, garment side surface of the backsheet, are secured to the bodyside surface of the opposed crotch portions 16, 24 of the first and second body panels 4, 6. It should be understood that in an alternative embodiment, the crotch member, for~example the body side thereof, can be secured to the garment side surface of the first and second body panels. It should be understood that the crotch member 50 can be secured using any of the methods of attachment described above, including for example various adhesives, stitching or other bonding methods. The crotch member can be secured to the body panels with any configuration of attachment lines, swirls, patterns, spots, etc., or can be a full and continuous attachment therebetween.

The entire portion of the crotch member 50 overlapping the body panels 4, 6 can be attached thereto, as shown in FIG. 4, with a continuous attachment 90. It should be understood that the various attachment configurations shown in FIGS. 4-4C, which illustrate the crotch member and front body panel, apply equally to the attachment configuration between the rear body panel and the crotch member. As shown in FIG. 4B, the crotch member 50 can be minimally attached to the body panels 4, 6, for example, by one or more lines of attachment 92 formed along the centerline of the absorbent insert 50. In yet another alternative embodiment, shown in FIG. 4C, the crotch member is attached to the body panels 4, 6 along outer longitudinal edges of the crotch member at attachment locations 94. In the embodiment of FIG. 4A, the crotch member 50 is attached along the outer edges thereof at locations 94 and also along a location 92, for example the centerline, therebetween. As shown in FIGS. 4A-4C, the outer lines of attachment 94 are spaced inboard of the outer edge of the crotch member 50 so as to form an unattached side margin 96, which functions as a flap while maximizing the ability of the underlying body panel to elongate and conform to the body of the user. The attachment locations 92 and 94 run the entirety of the length of the portion of the crotch member overlapping the corresponding body panel, although it should be understood that the attachment locations could be less than such a length, or comprise intermittent portions of attachment along each attachment location or line.

Referring to FIG. 5, a method and apparatus for fabricating one or more embodiments of the aforedescribed refastenable garments is illustrated. Referring to FIG. 5, one or more webs 100, 104, 106 of body panel material are moved in a longitudinal machine direction 102. The body panel material is elongated in the longitudinal machine direction to an elongated condition, for example by stretching the body panel material. In another embodiment, the body panel material is elongated in the lateral cross-direction. In one embodiment, the web is elongated a least 10% in the longitudinal machine direction. In one exemplary embodiment, the body panel material is elongated between about 25% and about 250%, and in another exemplary embodiment between about 50% and about 150%.

After the body panel material(s) 104, 106 are elongated in the longitudinal machine direction 102, the crotch member 50 is secured to the body panel material webs 104, 106 in one or more the attachment configurations and at the various attachment locations described herein, with the attachment locations extending in a lateral cross direction and being spaced apart in the longitudinal machine direction. The body panel material is then allowed to retract in the longitudinal machine direction. The retraction of the body panel material gathers the crotch 50 member in the longitudinal machine direction, especially when the crotch member is attached to the body panel material as shown in one of FIGS. 4, 4B and 4C. Alternatively, if the body panel material is elongated in the lateral cross direction, the crotch member is attached to thereto along one or more longitudinally extending locations that are laterally spaced in the cross direction, such the crotch member is gathered in the lateral cross direction.

In various embodiments, the method includes elongating front and rear body panel materials that are integral, or are formed at least in part from one piece. For example, as explained above, an outer cover can extend the length of the garment and can form one layer of each of the front and rear body panels. In another embodiment, a one-piece body chassis forms the entire front and rear body panels and a connecting crotch portion. In other embodiments, the front and rear body panels are discrete members and are separate and spaced apart in the lateral cross direction. The front and rear body panel materials, whether discrete or integral, can be elongated the same or different amounts in the percentage ranges disclosed above. The crotch member has opposite end portions attached to both of the body panel materials, as explained above, when they are in the elongated condition. The body panel materials are then allowed to retract or relax so as to thereby gather the crotch member in the direction of the elongation at both ends thereof.

Referring to FIG. 5, a single web 102 of body panel material can be cut in the longitudinal machine direction to form the front and rear body panel material web 104, 106. In one embodiment, the web 102 is cut in a sinusoidal wave pattern, which should be broadly interpreted as a pattern having peaks and valleys, with a cutter 108, which can be configured as a pair of drums, for example a knife and anvil roll. In various embodiments, the cut edges can be formed by an oscillating cutter, slitters, water jets, lasers and other known cutting devices. The pattern can be formed of undulating curves or wave patterns, or can include or be made entirely of various linear portions.

Each body panel web 102, 104 includes an outer lateral edge and an inner cut edge. In one embodiment, the inner cut edges of the front and rear body panel webs correspond, or mate such that they have the same shape and amplitude. In such an embodiment, no waste material is generated.

In one embodiment, one or both of the front and rear body panel material webs are shifted in the longitudinal machine direction 102, as shown to align the crotch portions of the body panel material webs. For example, as shown in FIG. 5, a first and second pivot conveyor 110, 112 can simply be spaced apart so as to provide for a longer travel for one of the front and rear body panel webs 104, 106. In various embodiments, the spacing between the cut edges at the maximum rise of the respective front and rear body panels (i.e., the closest spacing between the front and rear body panels) in one embodiment is between about 1% and about 90% of the total rise of the garment, in another embodiment between about 10% and about 60% of the total rise (or length) of the garment, and in another embodiment between about 20% and about 40% of the total rise of the garment. In addition, in various embodiments, the spacing between the cut edges at the maximum rise of the respective front and rear body panels is between about 10 mm and about 800 mm, between about 50 mm and about 500 mm, and between about 100 mm and about 300 mm. In an alternative embodiment, the cut edges and the crotch portions of the front and rear body panels overlap, and can be secured one to the other. In such an embodiment, the panels can be separated slightly, or can simply be shifted in the longitudinal direction without any lateral separation.

In one embodiment, the front and rear body panel webs 104, 106 are separated such that no portions of either web overlap each other. For example, and referring to FIG. 5, in one embodiment, pivot conveyors 110, 112 can be employed to spread the webs. In one embodiment, a first pair of rollers 114 can be angled or twisted to laterally spread the front and rear body panel webs 104, 106 a first amount before they are shifted in the longitudinal machine direction. A plurality of pairs of rollers 116 can be angled or twisted to laterally spread the front and rear body panel webs 104, 106 a second amount after they are shifted in the longitudinal machine direction. Of course, it should be understood that the front and rear body panels can be first shifted in the longitudinal machine direction the desired amount and then separated in the lateral cross direction the entire desired amount, or they can also be first separated in the lateral cross direction the entire desired amount and then shifted in the longitudinal machine direction. The body panel webs 104, 106 are put in tension and are elongated by the pairs of nip rollers 116. It should be understood that the webs can be put in tension at any number of different locations in the processing line. In addition, the front and rear body panels webs can be subject to different amounts of tension.

It should be understood that webs 104, 106 having an undulating cross-direction length, or a non-linear terminal crotch edge, will have a tension gradient formed across the length thereof in the cross direction at the peaks 118, 120. In other words, the machine direction tension exerted on the web is not uniform between the terminal edges 22, 28, 20, 14, and will drop off due to the discontinuity of the web in the machine direction. In this way, the body panel webs are not gathered as much, or at all, in the crotch region of the body panels after the body panels are relaxed with the crotch member secured thereto. Moreover, if the crotch member is secured to the body panel along at least a portion of the tension gradient, the body panel will be gathered lesser amounts in the area of lesser tension (and resultant elongation) upon relaxation of the body panel material. In this way, the crotch member will have a gathering gradient, with the crotch member being gathered in the lateral direction lesser amounts along its length.

As shown in FIG. 1, the crotch member 50, at least the end portions thereof, is secured to the body panels 4, 6 in an area where the body panel materials are in tension. In other embodiments, the elastic can be deadened in various selected regions to further control and limit the amount of tension and corresponding stretch and subsequent gathering of the body panel webs and body panels.

After the body panel webs 104, 106 are aligned and separated, regardless of the order thereof, a plurality of crotch members 50, for example absorbent inserts, are positioned in the lateral cross direction so as to bridge the gaps between the body panel webs at successive peaks 118, 120 (shown in FIG. 1) where the maximum rises of the body panel webs are aligned. It should be understood that the term “gap” as used herein includes a “zero” distance between the respective cut edges, wherein the cut edges abut but do not overlap. The crotch members 50 are secured to the body panel webs 104, 106 as explained above. It should be understood that the crotch members 50 are secured to a bodyside surface of the body panel webs, although they can also be secured to the garment side thereof. In one embodiment, the crotch members, for example the absorbent inserts, are assembled offline and are then applied to the front and rear body panel webs 76, 78 as those webs are carried by a construction drum 122.

As shown in FIG. 5, the crotch member is rotated using an offset cam action rotator 124. The rotator includes a plurality of transfer segments 126, which can have a vacuum applied thereto, that engage the crotch member 50. Coupler arms 127 connect the transfer segments and a drive ring. The coupler arm 127 includes a cam end having a cam follower that follows the profile of a cam mechanism. The profile of the cam mechanism can be readily changed to change the desired speed output and pitch of the crotch member. In one embodiment, the rotator is configured to accelerate the crotch member. If the successive crotch members 50 are separated by a perforation, the transfer segment 126 breaks the perforation as it engages one crotch member and moves away from the next crotch member, which is engaged by a next transfer segment 126. The rotator rotates the end portion of the transfer segment, for example approximately 90 degrees, about a radial axis, such that the crotch member is oriented in the machine direction as described above as the transfer segments are rotated about a horizontal axis 128. Alternatively, the landing material and fastener material are cut and separated by the transfer segments. The rotator, and the method for the use thereof, is further disclosed in U.S. Pat. Nos. 5,761,478, 5,759,340, and 6,139,004, and U.S. patent application Ser. No. 10/038,766, entitled “Apparatus For Applying Discrete Parts to A Moving Web,” filed Jan 2, 2002, all of which are assigned to Kimberly-Clark Worldwide, Inc., the assignee of the present application, and the entire disclosures of all of which are hereby incorporated herein by reference. Alternatively, the subassembly can be rotated using a revolving transfer roll as shown and described in U.S. Pat. No. 4,608,115, which is assigned to Kimberly-Clark Worldwide, Inc., the assignee of the present application, and which is hereby incorporated herein by reference in its entirety. In one embodiment, adhesive is applied to the crotch member 50 by an adhesive applicator 128 prior to the successive separation thereof by the rotator 124.

Referring to FIG. 5, after the crotch members 50 are secured to the elongated body panel material webs 104, 106 across the gap, the undergarments, and in particular the crotch members 50, are successively folded, using for example a helical folder, such that the front and rear body panel webs are positioned in an overlapping, or overlying relationship, for example with the outer edges aligned. In various alternative embodiments, the body panel webs can be secured, for example by bonding using a side seam bonder, to form the side seam. The front and rear body panel webs are then cut along the lateral cross direction along the seam to form a plurality of discrete disposable undergarments, each having a front and rear body panel 4, 6. As the body panel webs 104, 106 are cut, they are allowed to relax, thereby allowing the webs to retract and gather the crotch member 50. Alternatively, the front and rear body panel webs can be first cut, and the crotch member 50 thereafter folded. In yet another embodiment, the webs can be allowed to relax before the side seams are bonded.

Various refastenable fastening members can be applied to the front and rear body panels 4, 6 or front and rear body panel webs before or after the various cutting and folding operations. In one embodiment, as explained above, the undergarment can be configured with side seams which secure the front and rear body panels, and refastenable fastening members, which bridge lines of weakness formed in one or the other of the body panels.

In another aspect, the manufacturer or retailer of the afore-described absorbent garments provides instructional information to the user, for example by way of textual or pictorial indicia on the packaging materials, about how the garment works. For example, the manufacturer or retailer can explain to the end user the advantages of the gathered crotch member, and the resultant ability of the body panels to freely conform to the body of the user without restriction from the crotch member, thereby improving the fit and conformance of the garment.

Although the present invention has been described with reference to various exemplary embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

What is claimed is:

1. A method of manufacturing a disposable undergarment comprising:
elongating a body panel material in a first direction to an elongated condition;
attaching a crotch member to said body panel material while said body panel material is in said elongated condition; and
retracting said body panel material in said first direction from said elongated condition to a relaxed condition.

2. The method of claim 1 wherein said retracting said body panel material comprises gathering said crotch member in said first direction.

3. The method of claim 1 wherein said body panel material comprises a front body panel material and wherein said elongated and relaxed conditions comprise first elongated and relaxed conditions respectively, and further comprising a rear body panel material spaced apart from said front body panel material in a second direction, and further comprising elongating said rear body panel material in said first direction to a second elongated condition, attaching said crotch member to said rear body panel material while said rear body panel material is in said second elongated condition, and retracting said rear body panel material in said first direction from said second elongated condition to a second relaxed condition.

4. The method of claim 3 wherein said retracting said rear body panel material comprises gathering said crotch member in said first direction.

5. The method of claim 1 wherein said crotch member has a length, opposite end portions and opposite side edges, wherein at least one of said opposite end portions overlies said body panel material, and wherein said attaching said crotch member to said body panel material comprises attaching said end portion of said crotch member overlying said body panel material to said body panel material.

6. The method of claim 5 wherein said attaching said end portion of said crotch member to said body panel material comprises attaching said end portion to said body panel material at a plurality of attachment locations spaced apart in said first direction.

7. The method of claim 6 wherein said plurality of attachment locations comprises at least a pair of attachment locations positioned adjacent said opposite side edges of said crotch member.

8. The method of claim 7 wherein said at least said pair of attachment locations run substantially an entirety of said end portion that overlies said crotch member.

9. The method of claim 6 wherein said plurality of attachment locations comprises at least a pair of attachment locations positioned inboard from said opposite side edges of said crotch member, wherein said opposite side edges form opposite unattached flaps overlying said body panel material.

10. The method of claim 5 wherein said attaching said end portion of said crotch member to said body panel material comprises attaching said end portion to said body panel material at an attachment location located substantially along a centerline of said crotch member.

11. The method of claim 10 wherein said attachment location has a width less than a width of said crotch member measured between said opposite sides thereof.

12. The method of claim 1 wherein said crotch member comprises an absorbent insert.

13. The method of claim 1 wherein said elongating said body panel material in said first direction to said elongated condition comprises elongating said body panel material at least about 10% in said first direction.

14. The method of claim 13 wherein said elongating said body panel material at least 10% in said first direction comprises elongating said body panel material between about 25% and about 250% in said first direction.

15. The method of claim 14 wherein said elongating said body panel material between about 25% and about 250% in said first direction comprises elongating said body panel material between about 50% and about 150% in said first direction.

16. The method of claim 1 wherein said attaching said crotch member to said body panel material comprises attaching said crotch member to a bodyside surface of said body panel material.

17. The method of claim 1 wherein said attaching said crotch member to said body panel material comprises attaching said crotch member to a garment side surface of said body panel material.

18. A method of manufacturing a disposable undergarment comprising:
elongating a first body panel material in a first direction to a first elongated condition;
elongating a second body panel material in said first direction to a second elongated condition;
attaching an absorbent insert to said first and second body panel materials at a plurality of attachment locations while said first and second body panel materials are in said first and second elongated conditions, wherein said attachment locations are spaced apart in said first direction; and
retracting said first and second body panel materials in said first direction from said first and second elongated conditions to first and second relaxed conditions respectively.

19. The method of claim 18 wherein said retracting said first and second body panel materials comprises gathering said absorbent insert in said first direction.

20. The method of claim 18 wherein said absorbent insert has a length, first and second end portions and opposite side edges, wherein said first and second end portions overlie respectively said first and second body panel materials, and wherein said attaching said absorbent insert to said first and second body panel materials comprises attaching said first end portion to said first body panel material at a plurality of first attachment locations spaced apart in said first direction and attaching said second end portion to said second body panel material at a plurality of second attachment locations spaced apart in said first direction.

21. The method of claim 20 wherein said pluralities of first and second attachment locations comprises at least a pair of said first attachment locations positioned inboard from said opposite side edges of absorbent insert and at least a pair of said second attachment locations positioned inboard from said opposite side edges of absorbent insert, wherein said opposite side edges form opposite unattached flaps overlying said first and second body panel materials.

22. The method of claim 20 wherein said elongating said first and second body panel materials in said first direction to said first and second elongated conditions respectively comprises elongating said first and second body panel materials at least about 10% in said first direction.

23. The method of claim 22 wherein said elongating said first and second body panel materials at least 10% in said first direction comprises elongating said first and second body panel materials between about 25% and about 250% in said first direction.

24. The method of claim 23 wherein said elongating said first and second body panel materials between about 25% and about 250% in said first direction comprises elongating said first and second body panel materials between about 50% and about 150% in said first direction.

25. A disposable undergarment comprising:
a body panel elongateable in a first direction between at least a relaxed condition and an elongated condition; and
a crotch member attached to said body panel and elongateable in said first direction between at least a gathered condition and an ungathered condition, wherein said crotch member is in said gathered condition when said body panel is in said relaxed condition, and wherein said crotch member is in said ungathered condition when said body panel is in said elongated condition.

26. The disposable undergarment of claim 25 wherein said body panel comprises a front body panel and wherein said elongated and relaxed conditions comprise first elongated and relaxed conditions respectively, and further comprising a rear body panel spaced apart from said front body panel material in a second direction, and wherein said crotch member is attached to said rear body panel, wherein said crotch member is in said gathered condition when said rear body panel is in said relaxed condition, and wherein said crotch member is in said ungathered condition when said rear body panel is in said elongated condition.

27. The disposable undergarment of claim 26 wherein said crotch member has a length, first and second end portions and opposite side edges, wherein said first and second end portions overlie respectively said first and second body panels, and wherein said first end portion is attached to said front body panel at a plurality of first attachment locations spaced apart in said first direction and wherein said second end portion is attached to said rear body panel at a plurality of second attachment locations spaced apart in said first direction.

28. The disposable undergarment of claim 27 wherein said pluralities of first and second attachment locations comprises at least a pair of said first attachment locations positioned inboard from said opposite side edges of absorbent insert and at least a pair of said second attachment locations positioned inboard from said opposite side edges of absorbent insert, wherein said opposite side edges form opposite unattached flaps overlying said first and second body panel materials.

29. The disposable undergarment of claim 25 wherein said body panel is elongated at least about 10% in said first direction when in said elongated condition.

30. The disposable undergarment of claim 29 wherein said body panel is elongated between about 25% and about 250% in said first direction when in said elongated condition.

31. The disposable undergarment of claim 29 wherein said body panel is elongated between about 50% and about 150% in said first direction when in said elongated condition.

32. The disposable undergarment of claim 25 wherein said crotch member is attached to said body panel material at an attachment location located substantially along a centerline of said crotch member.

33. The disposable undergarment of claim 32 wherein said attachment location has a width less than a width of said crotch member measured between opposite sides thereof.

34. The disposable undergarment of claim 25 wherein said crotch member comprises an absorbent insert.

35. The disposable undergarment of claim 25 wherein said crotch member is attached to a bodyside surface of said body panel.

36. The disposable undergarment of claim 35 wherein said crotch member is attached to a garment side surface of said body panel.

37. A method of using a disposable undergarment comprising:
providing an enlongateable body panel in a relaxed condition and a crotch member attached to said body panel, wherein said crotch member is in a gathered condition when said body panel is in said relaxed condition; and
elongating said body panel in a first direction from said relaxed condition to an elongated condition and thereby elongating said gathered crotch member in said first direction.

38. The method of claim 37 wherein said body panel comprises a front body panel and wherein said elongated and relaxed conditions comprise first elongated and relaxed conditions respectively, and further comprising providing a rear body panel spaced apart from said front body panel in a second direction and attached to said crotch member, and further comprising elongating said rear body panel in said first direction from a second relaxed condition to a second elongated condition.

39. The method of claim 37 wherein said crotch member is attached to said body panel at a plurality of attachment locations spaced apart in said first direction.

40. The method of claim 39 wherein said plurality of attachment locations comprises at least a pair of attachment locations positioned adjacent opposite side edges of said crotch member.

41. The method of claim 39 wherein said plurality of attachment locations comprises at least a pair of attachment locations positioned inboard from opposite side edges of said crotch member, wherein said opposite side edges form opposite unattached flaps overlying said body panel.

42. The method of claim 37 wherein said crotch member is attached to said body panel at an attachment location located substantially along a centerline of said crotch member.

43. The method of claim 42 wherein said attachment location has a width less than a width of said crotch member measured between opposite sides thereof.

44. The method of claim 37 wherein said crotch member comprises an absorbent insert.

45. The method of claim 37 wherein said elongating said body panel in said first direction to said elongated condition comprises elongating said body panel at least about 10% in said first direction.

46. A method of instructing a user on the use of a disposable undergarment comprising:
providing an enlongateable body panel in a relaxed condition and a crotch member attached to said body panel, wherein said crotch member is in a gathered condition when said body panel is in said relaxed condition; and
instructing a user to apply said undergarment to the body of the user.

47. The method of claim 46 further comprising advising the user that the application of the undergarment to the body of the user will elongate said body panel in a first direction from said relaxed condition to an elongated condition and thereby elongate said gathered crotch member in said first direction.
